(19) United States
(12) Patent Application Publication
(10) Pub. No.: US 2010/0009997 A1
(43) Pub. Date: Jan. 14, 2010

(54) METHOD OF TREATING A METABOLIC OR NEUROPSYCHIARTY DISORDER WITH A BH4 DERIVATIVE PRODRUG

(75) Inventors: Daniel I. Oppenheimer, Castro Valley, CA (US); Emil D. Kakkis, Novato, CA (US); Alejandro Dorenbaum, Mill Valley, CA (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: BIOMARIN PHARMACEUTICAL INC., Novato, CA (US)

(21) Appl. No.: 12/519,806

(22) PCT Filed: Jan. 14, 2008
(86) PCT No.: PCT/US2008/051009

§ 371 Date: Sep. 17, 2009

Related U.S. Application Data
(60) Provisional application No. 60/884,721, filed on Jan. 12, 2007.

(51) Int. Cl. A61K 31/519; A61P 25/00

(52) U.S. Cl. 514/249

(57) ABSTRACT

Low dose therapeutic methods for use of BH4 derivatives to treat BH4-responsive disorders, such as hyperphenylalanemia and neuropsychiatric diseases, and combination therapies of BH4 derivatives and other therapeutic regimens, are described.

### CROSS REFERENCE TO RELATED APPLICATIONS

This application is a continuation of U.S. Provisional Application No. 60/884,721, which was filed on Jan. 12, 2007. The entire disclosure of this priority application is hereby incorporated herein by reference.

### BACKGROUND

1. Field of the Invention

The present invention is generally directed to the therapeutic intervention of BH4-responsive diseases using BH4 derivatives at low doses.

2. Background of the Related Art

Tetrahydrobiopterin (also referred to herein as “BH4”) is a naturally-occurring chemical compound having the chemical structure shown in Formula I, below, and is a biologically active amine of the pterin family:

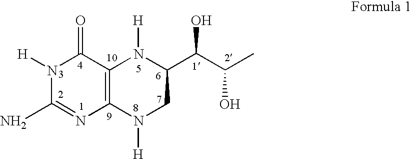

Tetrahydrobiopterin functions as a cofactor for a number of different enzymes, including phenylalanine hydroxylase (PAH), tyrosine 3-hydroxylase, tryptophan 5-hydroxylase, and all three forms of nitric oxide synthase (NOS). These and other cofactor and cellular functions of tetrahydrobiopterin as well as disorders relating to tetrahydrobiopterin deficiency are disclosed in Thony et al. (2000) Biochem. J. 347:1-16. Disorders relating to tetrahydrobiopterin deficiency also are generally described in Blau et al., Disorders of Tetrahydrobiopterin and Related Biogenic Amines, in The Metabolic and Molecular Bases of Inherited Disease, 1275-776 (8th ed., McGraw-Hill Publishing Co., New York, N.Y., 2001).

There remains a need for new therapeutic modalities for diseases in which BH4 may play a role.

### SUMMARY

The invention provides for the administration of a BH4 derivative prodrug, such as a diacyl or diacetyl BH4, or a lipoidal BH4 as described herein, at an unexpectedly low dose that still achieves therapeutic efficacy. In one embodiment, it is contemplated that the BH4 derivative has better intestinal absorption and/or higher oral bioavailability than BH4 and therefore can be administered at a lower dose than BH4 to achieve the same efficacy. Thus, the therapeutically efficacious doses of such BH4 prodrugs will be lower than the conventional dose of BH4 for treatment of phenylketonuria. Such low dose therapeutic methods are applicable to any BH4-responsive disease, including BH4 deficiency, neuropsychiatric disorders, preferably those that would benefit from increased tyrosine hydroxylase function or tryptophan hydroxylase function, and metabolic disorders, including hyperphenylalanemia, or mild, moderate and severe phenylketonuria, and optionally excluding vascular diseases and diseases related to nitric oxide synthase dysfunction.

In exemplary embodiments, the BH4 derivative is an ester form, wherein ester groups are added in diester form at the vicinal hydroxyl sites in the BH4 molecule. Ester groups include but are not limited to formyl, acetyl, propyl, and butyral groups. In one embodiment, the ester prodrug is a dibutyral ester form of BH4.

In a related aspect, the invention contemplates that the subject additionally can be treated with one or more other agents that (a) enhance the activity or expression or the de novo biosynthesis of BH4, and/or (b) reduce the degradation of BH4 or BH4 derivatives and/or (c) stabilize BH4 or BH4 derivatives. Exemplary agents can be selected from the group consisting of guanosine triphosphate cyclohydrolase I (GTPCH1), 6-pyruvoyltetrahydropterin synthase (PTPS) and sepiapterin reductase. In a preferred embodiment of the invention, BH4 synthesis is increased by increasing the expression of GTPCH1 expression by the use of any one or more cyclic adenosine monophosphate (cAMP) analogs or agonists including forskolin, 8-bromo cAMP or other agents that function to increase cAMP mediated cell signaling, for example, cytokines and growth factors including interleukin-1, interferon-gamma (IFN-γ), tumor necrosis factor alpha (TNF-α), c-reactive protein, HMG-CoA-reductases (statins like atorvastatin) nerve growth factor (NGF), epidermal growth factor (EGF), hormones including adrenomedullin and estradiol benzoate, and other compounds such as NADPH and NADPH analogs, caffeine, cyclosporine A methyl-xanthines including 3-isobutyl-1-methyl xanthine, theophylline, reserpine, hydrogen peroxide.

In another optional aspect, the invention contemplates co-administration of an agent that increases GTPCH1 levels by inhibiting the degradation of 3′5′-cyclic nucleotides using inhibitors of the eleven phosphodiesterase families (PDE1-11) including PDE1, PDE3, PDE5. The PDE inhibitors of the present invention include Viagra/sildanafil, cialis/tadalafil, vardenafil/levitra, udenafil, 8-Methoxymethyl-IBMX, UK-90234, dexamethasone, hesperetin, hesperedins, Irsogladine, vinpocetine, cilostamide, rolipram, ethyl beta-carboline-3-carboxylate (beta-CCE), tetrahydro-beta-carboline derivatives, 3-O-methylquercetin and the like.

In another non-exclusive option, the levels of BH4 can be increased by increasing the levels of BH4-synthesizing enzymes by gene therapy or endothelium-targeted delivery of polynucleotides of the synthetic machinery of BH4. Yet another embodiment of the invention relates to increasing the levels of BH4 by supplementation with BH4-synthesizing enzymes GTPCH1, PTPS, SR, PCD, DHPR and DHFR. It is contemplated that BH4-synthesizing enzymes encompass all natural and unnatural forms of the enzymes including mutants of the proteins.

Another non-exclusive optional aspect of the invention relates to co-administration of an agent that increases BH4 levels by diverting the substrate 7,8-dihydroneopterin triphosphate towards BH4 synthesizing enzyme PTPS instead of alkaline phosphatase (AP) by inhibiting AP activity. Agents that inhibit the activity of AP include phosphate analogs, levamisole, and L-Phe. In another embodiment, agents that inhibit AP include small inhibitory RNA (siRNA), antisense RNA, dsDNA, small molecules, neutralizing antibodies, single chain, chimeric, humanized and antibody fragments to inhibit the synthesis of alkaline phosphatase.

In yet another non-exclusive option, agents that enhance the activity of catalysts or cofactors needed for the synthesis of enzymes of the de novo synthesis pathway of BH4 synthesis can be co-administered with the BH4 derivatives.

Still another non-exclusive optional aspect of the invention includes co-administration of agents that prevent the degradation of the enzymes needed for the synthesis of BH4. Yet another non-exclusive optional aspect of the invention includes co-administration of agents that prevent the degradation of the catalysts needed for the synthesis of BH4 and its synthetic enzymes including GTPCH1, PTPS and SR.

Another non-exclusive optional embodiment of the invention relates to co-administration of agents that increase the levels of BH4 by increasing the reduction of BH2 via the salvage pathway. In vivo, BH4 becomes oxidized to BH2. BH2 which exist as the quinoid form (qBH2) and as the 7,8-dihydropterin which is reduced to BH4 by DHPR and DHFR respectively. A preferred embodiment of the invention relates to increasing the regeneration or salvage of BH4 from BH2 by modulating the activity and synthesis of the enzymes PCD, DHPR and DHFR using agents of that pathway, such as NADPH, thiols, perchloromercuribenzoate, hydrogen peroxide and the like.

Another non-exclusive optional aspect of the invention relates to co-administration of agents that stabilize BH4 by decreasing the oxidation of BH4 using agents or compounds such as antioxidants including ascorbic acid (vitamin C), alpha tocopherol (vitamin E), tocopherols (e.g vitamin A), selenium, beta-carotenes, carotenoids, flavones, flavonoids, folates, flavones, flavanones, isoflavones, catechins, anthocyanidins, and chalcones.

In yet another non-exclusive embodiment, the invention contemplates co-administration of factors that inhibit the GTPCH feedback regulatory protein, GFRP. A preferred embodiment of the invention relates to agents that inhibit the binding of BH4 to the GTPCH1/GFRP complex, thereby preventing the feedback inhibition by BH4. Such agents include competitive inhibitors such as alternate forms of BH4 with altered affinities for the complex, structural analogs etc. Still another embodiment of the invention includes co-administration of agents that enhance the binding of L-phenylalanine to GTPCH1/GFRP inducing the synthesis of BH4. Another embodiment of the invention includes co-administration of agents that increase the levels of L-Phe such as precursors of L-Phenylalanine, which serves to inhibit the feedback inhibition of GTPCH1 by GFRP and BH4.

Yet another non-exclusive embodiment of the invention relates to co-administration of agents that modulate the activity or the synthesis of GFRP. A preferred embodiment of the invention includes agents that inhibit the activity of GFRP. Another non-exclusive embodiment of the invention includes the use of siRNA, small molecules, antibodies, antibody fragments and the like to inhibit the synthesis of GFRP.

The invention further contemplates co-administration of precursors of BH4 including guanosine triphosphate, 7,8-dihydro-neopterin triphosphate and 6-pyrovoyl tetrahydropbiopterin.

In other non-exclusive embodiments, the BH4 prodrug derivatives may be co-administered with folates, including folate precursors, folic acids, and folate derivatives. Such folates include but are not limited to tetrahydrofolate, 5-formyl-(6S)-tetrahydrofolic acid and salts thereof, 5-methyl-(6S)-tetrahydrofolic acid and salts thereof, 5,10-methylene-(6R)-tetrahydrofolic acid and salts thereof, 5,10-methenyl-(6R)-tetrahydrofolic acid and salts thereof, 10-formyl-(6R)-tetrahydrofolic acid, 5-formimino-(6S)-tetrahydrofolic acid and salts thereof, (6S)-tetrahydrofolic acid and salts thereof, and combinations of the foregoing. In a further embodiment, the BH4 prodrugs may be additionally co-administered with arginine.

The BH4 prodrugs may be administered in a single daily dose or in multiple doses on a daily basis, and are preferably administered in total daily doses of at least 0.1 mg/kg and preferably less than 10 mg/kg, 5 mg/kg or less, 4 mg/kg or less, or 3 mg/kg or less, for example in ranges of 0.1 mg/kg to 5 mg/kg, or 0.1 mg/kg to 4 mg/kg, or 0.1 to 3 mg/kg. In some embodiments, the BH4 derivative therapy is not continuous, but rather is administered on a daily basis until improvement in clinical endpoints (e.g. reduction in serum Phe levels, or normalization of neurological symptoms,) is maintained.

Also contemplated is use of a BH4 prodrug in preparation of a medicament for the treatment of a disorder as disclosed herein comprising administration of the BH4 prodrug according to any of the methods as disclosed herein. It is understood that co-administration methods involving administration of BH4 prodrug with a second therapeutic agent, as described herein, encompass the use of the second therapeutic agent in preparation of a medicament for co-administration with a BH4 prodrug as disclosed herein.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### DESCRIPTION OF THE PREFERRED EMBODIMENTS

### I. BH4 Derivatives in Treatment

One embodiment of the invention entails administering a BH4 derivative composition to any individual with abnormal endpoints in an amount effective to normalize values. In some embodiments, the individual to be treated is diagnosed with hyperphenylalanemia or a neuropsychiatric disorder.

Those of skill in the art will understand that the invention contemplates treating patients having typical symptoms with BH4 to produce normal values for clinically relevant endpoints. Further, any changes in endpoint values within the minimal of normal ranges for clinically relevant endpoints will be considered a therapeutic outcome for the therapeutic regimens for the patients.

### 1. BH4 and the Treatment of Neuropsychiatric Disorders

NO overproduction by nNOS has been implicated in strokes, migraine headaches, Alzheimer's disease, and with tolerance to and dependence on morphine. BH4 derivatives may be administered for any of these conditions. Other exemplary neuropsychiatric disorders for which BH4 derivatives may be administered include Parkinson's disease, Alzheimer's disease, schizophrenia, schizophreniform disorder, schizoaffective disorder, brief psychotic disorder, delusional disorder, shared psychotic disorder, psychotic disorder due to a general medical condition, substance-induced psychotic disorder, other psychotic disorders, tardive dyskinesia, Machado-Joseph disease, spinocerebellar degeneration, cerebellar ataxia, dystonia, chronic fatigue syndrome, acute or chronic depression, chronic stress syndrome, fibromyalgia, migraine, attention deficit hyperactivity disorder, bipolar disease, and autism. The neuropsychiatric disorder may be associated with reduced tyrosine hydroxylase function or reduced tryptophan hydroxylase function. Neuropsychiatric disorders herein optionally exclude Parkinson's disease, depression, and Alzheimer's disease.

BH4 derivatives may be co-administered according to the method of the invention with one or more other neuropsychiatric active agents, including antidepressants, neurotransmitter precursors such as tryptophan, tyrosine, serotonin, agents which activate noradrenergic systems, such as lofepramine, desipramine, reboxetine, tyrosine, agents which act preferentially on serotonin, combined inhibitors of both noradrenaline and serotonin uptake, such as venlafaxine, duloxetine or milnacipran, and drugs which are combined inhibitors of both dopamine and noradrenaline reuptake such as bupropion.

In exemplary embodiments, the amount of BH4 or precursor or derivative administered increases tyrosine hydroxylase function or tryptophan hydroxylase function by at least 5, 10, 15, 20, 25, 30, 35, 40, 50, 75, or 100%, or increases neurotransmitter levels of L-Dopa or serotonin by at least 5, 10, 15, 20, 25, 30, 40, 50, 75, or 100% in BH4-responsive patients.

### 2. BH4 and the Treatment of Metabolic Disorders Such as HPA

Exemplary metabolic disorders include hyperphenylalanemia, e.g., mild phenylketonuria, classic phenylketonuria, severe phenylketonuria, atypical or malignant phenylketonuria associated with BH4 deficiency, hyperphenylalanemia associated with liver disorder, and hyperphenylalanemia associated with malaria. Exemplary patient populations include infants, children, teenagers, adults, females of childbearing age, and pregnant females. In some embodiments, the individual has a plasma phenylalanine concentration of greater than 1000 μM in the absence of treatment with (e.g., pre-treatment) the BH4 prodrug, and administration of the compound is in an amount effective to decrease the plasma phenylalanine concentration in the individual to less than about 1000 μM, or less than about 800 μM, or less than about 700 μM, or less than about 600 μM, or less than about 500 μM, or less than about 450 μM, ±15 μM.

In exemplary embodiments, the subject with hyperphenylalaninemia or phenylketonuria has been diagnosed as having a mutant phenylalanine hydroxylase (PAH). The mutant PAH may comprise a mutation in the catalytic domain of PAH. Exemplary such mutations include one or more mutations selected from the group consisting of F39L, L48S, I65T, R68S, A104D, S110C, D129G, E178G, V190A, P211T, R241C, R261Q, A300S, L308F, A313T, K320N, A373T, V388M E390G, A395P, P407S, and Y414C. In other exemplary embodiments, the subject has an above normal concentration of plasma or serum phenylalanine (e.g., greater than 180 μM and, more preferably, greater than 360 μM) when untreated. In exemplary embodiments, the plasma or serum phenylalanine concentration is greater than 180 μM but less than 600 μM, or greater than 500 μM but less than 1200 μM, or greater than 1000 μM, or between 120 μM and 200 μM, or between 200 μM and 600 μM, or between 600 μM and 1200 μM, or greater than 1200 μM. In specific embodiments, the patient is an infant, more particularly, an infant having a plasma phenylalanine concentration greater than 1200 μM. In other embodiments, the patient is pregnant and pregnant patient has a plasma phenylalanine concentration of between about 200 μM to about 600 μM. Pregnant patients with a plasma phenylalanine concentration greater than 1200 μM are particularly attractive candidates for this type of therapy, as are patients who are females of child-bearing age that are contemplating pregnancy.

Patients suffering from hyperphenylalaninemia, including phenylketonuria, are frequently treated with a protein-restricted diet, typically a phenylalanine-restricted diet wherein the total phenylalanine intake of the subject is restricted to less than 600 mg per day. Co-treatment with a BH4 derivative and a protein-restricted diet is contemplated according to the invention. In embodiments, the protein-restricted diet is a phenylalanine-restricted diet wherein the total phenylalanine is restricted to less than 300 mg per day. In still other embodiments, the protein-restricted diet is one which is supplemented with amino acids, such as tyrosine, valine, isoleucine and leucine. The patient may be co-administered a low-Phe protein supplement, which may include L-tyrosine, L-glutamine, L-carnitine at a concentration of 20 mg/100 g supplement, L-taurine at a concentration of 40 mg/100 g supplement and selenium. It may further comprise the recommended daily doses of minerals, e.g., calcium, phosphorus and magnesium. The supplement further may comprise the recommended daily dose of one or more amino acids selected from the group consisting of L-leucine, L-proline, L-lysine acetate, L-valine, L-isoleucine, L-arginine, L-alanine, glycine, L-asparagine monohydrate, L-tryptophan, L-serine, L-threonine, L-histidine, L-methionine, L-glutamic acid, and L-aspartic acid. In addition, the supplement may be fortified with the recommended daily dosage of vitamins A, D and E. Optionally, the supplement comprises a fat content that provides at least 40% of the energy of the supplement. Such supplements may be provided in the form of a powder supplement or in the form of a protein bar. In certain embodiments, protein-restricted diet comprises a protein supplement and the BH4 is provided in the same composition as the protein supplement.

### II. BH4 Dosing and Compositions

In exemplary embodiments, it is contemplated that the methods of the present invention will provide to a patient in need thereof, a daily dose of less than 10 mg/kg of BH4 prodrug. Of course, one skilled in the art may adjust this dose up or down depending on the efficacy being achieved by the administration. The daily dose may be administered in a single dose or alternatively may be administered in multiple doses at conveniently spaced intervals. In exemplary embodiments, the daily dose may be about 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

In the low dose therapeutic methods of the invention, low doses, e.g., doses of 0.1 to 5 mg/kg are contemplated, including doses of 0.1 to 2 mg/kg or of 1 mg/kg to 5 mg/kg. Such doses are expected to provide improvements with relevant study endpoints. In particular, the invention contemplates that any of the 1′,2′-diacyl-(6R,S)-5,6,7,8-tetrahydro-L-biopterins or lipoidal tetrahydrobiopterins described below are particularly useful at low doses because of increased oral bioavailability.

For the person of average weight/body surface area (e.g. 70 kg), the invention also contemplates a total daily dose of less than 400 mg. Exemplary such total daily doses include 360 mg/day, 350 mg/day, 300 mg/day, 280 mg/day, 210 mg/day, 180 mg/day, 175 mg/day, 150 mg/day, or 140 mg/day. For example, 350 mg/day or 175 mg/day is easily administrable with an oral dosage formulation of 175 mg, once or twice a day. Other exemplary total daily doses include 320 mg/day or less, 160 mg/day or less, or 80 mg/day or less. Such doses are easily administrable with an oral dosage formulation of 80 or 160 mg. Other exemplary total daily doses include 45, 90, 135, 180, 225, 270, 315 or 360 mg/day or less, easily administrable with an oral dosage formulation of 45 or 90 mg. Yet other exemplary total daily doses include 60, 120, 180, 240, 300, or 360 mg/day, easily administrable with an oral dosage formulation of 60 or 120 mg. Other exemplary total daily doses include 70, 140, 210, 280, or 350 mg/day, easily administrable with an oral dosage formulation of 70 or 140 mg. Exemplary total daily doses also include 55, 110, 165, 220, 275 or 330 mg/day, easily administrable with an oral dosage formulation of 55 mg. Other exemplary total daily doses include 65, 130, 195, 260, or 325 mg/day, or 75, 150, 225, 300 or 375 mg/day, e.g. in dosage formulations of 65 mg or 75 mg.

### 1. Combination Therapy

The present invention further contemplates the therapeutic intervention of various types of BH4-related dysfunction by administration of BH4 alone or in combination with one or more agent or one or more intervention also used to treat that dysfunction. It should be understood that the BH4 therapies may be combined with conventional agents or interventions to treat dysfunctions to effect the therapeutic increase in clinically relevant endpoints for such disease in such patients. As described above, treatment of BH4-related dysfunction is directed at maintaining homeostasis, providing adjuvant therapy and providing specific therapy to improve clinical relevant endpoints. Homeostasis is maintained by correcting factors that predispose to BH4-related dysfunctions including levels of BH4 without increasing the propagation of the dysfunction. Adjuvant therapy consists of administering agents or interventions that increase the effectiveness of the primary therapy. Specific therapy is directed at maintaining normal clinical relevant endpoints. The methods and compositions of the invention contemplate pharmaceutical compositions of the BH4 prodrug that may be delivered through any conventional route of administration, including but not limited to oral, intramuscular injection, subcutaneous injection, intravenous injection and the like. The compositions of the present invention may further comprise a BH4 prodrug in combination with an antioxidant that aids in prolonging the stability of the BH4 prodrug composition.

Optional methods within the invention involve the combined use (“co-administration”) of BH4 and one or more conventional agents and/or one or more interventions to effect a therapeutic outcome in patients with BH4-related dysfunction. To achieve the appropriate therapeutic outcome in the combination therapies contemplated herein, one would generally administer to the subject the BH4 prodrug and the agents/intervention in a combined amount effective to produce the desired therapeutic outcome. This process may involve administering the BH4 prodrug and the agent/intervention at the same time. This may be achieved by administering a single composition or pharmacological formulation that includes both the therapeutic agent and the BH4 prodrug in a combined dosage form or administering the BH4 prodrug at the same time as the interventions is being conducted. Alternatively, the agent/intervention is taken at about the same time as a pharmacological formulation (tablet, injection or drink) of the BH4 prodrug. In other alternatives, the BH4 prodrug treatment may precede or follow the agent/intervention by intervals ranging from minutes to hours to days. In embodiments wherein the agent/intervention and the BH4 prodrug are administered separately, one would generally ensure that both the agent/intervention and BH4 are exerting their effect concurrently, such that the BH4 will still be able to exert an advantageous effect on the patient. In such instances, it is contemplated that one would administer the BH4 prodrug within about 2-6 hours (before or after) of the agent/intervention, with a delay time of only about 1 hour being most preferred. However, it should be understood the 2-6 hour time frame between administration of the two is merely exemplary, it may be that longer time intervals, e.g., 24 hours, 36 hours, 48 hours, 72 hours, one week or more between administration of the BH4 prodrug and the second agent/intervention also is contemplated. In certain embodiments, it is contemplated that the BH4 prodrug therapy will be a continuous therapy where a daily dose of BH4 prodrug is administered to the patient indefinitely.

### 2. BH4 Prodrug Compositions for Use in Therapy, Including Low Dose Therapy

The present section provides a description of the compositions that may be used in the treatments contemplated herein.

U.S. Pat. Nos. 5,698,408; 2,601,215; 3,505,329; 4,540,783; 4,550,109; 4,587,340; 4,595,752; 4,649,197; 4,665,182; 4,701,455; 4,713,454; 4,937,342; 5,037,981; 5,198,547; 5,350,851; 5,401,844; 5,698,408 and Canadian application CA 2420374 (each incorporated herein by reference) each describe methods of making dihydrobiopterins, BH4 and derivatives thereof that may be used as compositions for the present invention. Any such methods may be used to produce compounds for prodrugs for use in the therapeutic methods of the present invention.

In particular, U.S. Pat. No. 4,540,783 describes BH4 derivatives that are 1′,2′-diacyl-(6R,S)-5,6,7,8-tetrahydro-L-biopterins, and inorganic or organic salts thereof, that are useful according to the therapeutic methods of the invention. Preferably pharmaceutically acceptable salts are used for therapeutic methods of the invention. The compounds described in U.S. Pat. No. 4,540,783 have the general formula (I):

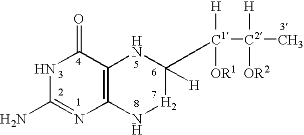

wherein R¹ and R² are the same or different and each is an acyl group.

The acyl group has preferably 1 to 10 carbon atoms, in particular 3 to 10 carbon atoms. Preferably, the acyl group is represented by the general formula R⁵CO— wherein R⁵ is hydrogen or a hydrocarbon residue having 1 or more carbon atoms, in particular 2 to 9 carbon atoms. Preferable examples of the hydrocarbon residue represented by R⁵ are, for instance, a linear or branched alkyl group having 1 or more carbon atoms, preferably 2 to 9 carbon atoms, which is either saturated or unsaturated; a substituted or unsubstituted phenyl group represented by the general formula

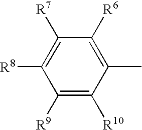

wherein R⁶, R⁷, R⁸, R⁹ and R¹⁰ are hydrogen or a linear or branched alkyl group wherein the combined number of carbon atoms is R⁶, R⁷, R⁸, R⁹, R¹⁰ is preferably not more than 3; a substituted or unsubstituted benzyl group represented by the general formula

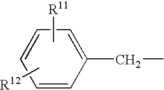

wherein R¹¹ and R¹² are hydrogen, methyl or ethyl wherein the combined number of carbon atoms R¹¹ and R¹² is preferably not more than 2; and a substituted or unsubstituted arylalkyl group represented by the general formula

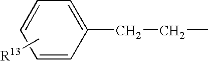

wherein R¹³ is hydrogen or methyl group. Among the above acyl groups, formyl, acetyl, propionyl, butyryl, isobutyryl, valeryl, isovaleryl and benzoyl are most preferable. It is preferable that R¹ and R² are the same.

The compound of the general formula (I) has two diastereomers, i.e. 1′,2′-diacyl-(6R)-5,6,7,8-tetrahydro-L-biopterin and 1′,2′-diacyl-(6S)-5,6,7,8-tetrahydro-L-biopterin which are diastereomeric at the 6 position. The compound for use in the invention can include either of the two diastereomers or a mixture thereof.

The compound (I) of the present invention can be in a form of an inorganic salt such as a hydrochloride, a sulfate or a phosphate, an organic salt such as an acetate, an oxalte, or a complex salt.

U.S. Pat. No. 4,550,109 describes BH4 derivatives that are lipoidal biopterins and tetrahydrobiopterins. These lipoidal BH4 derivatives may be administered as pharmaceutically acceptable salts according to the therapeutic methods of the invention. The compounds described in U.S. Pat. No. 4,550,109 are represented by the following structure:

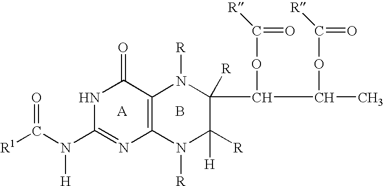

wherein

R is absent when there are two double bonds in ring B;

R is hydrogen when the two double bonds in ring B are absent; and

R′ and R″ are independently saturated or unsaturated, aliphatic hydrocarbon groups which are balanced in molecular weight such that they confer to the lipoidal compound a lipoidal property.

Generally R′ and R″ are selected from hydrocarbons having from 1 to 31 carbon units, with the limitation that the sum of carbon units of R′+2R″ is greater than 10 but less than 33.

In these derivatives, the 2-N-acyl group is desirably from 9 to 32 and preferably 9 to 20 carbon units so as to confer lipoidal characteristics upon the final product. The 2-N-acyl group is exemplified by decanolyl-, palmitoyl-, stearoyl- and linoleyl. The 2-N-acyl group may be saturated as is stearoyl- or unsaturated as is linoleyl. In addition, non-toxic aromatic 2-N-acyl groups like phenylacetyl can also confer the desirable lipoidal characteristics to the final product. The 1′,2′-di-O-acyl groups, are desirably lower molecular weight alkyls and alkenyls having from 2 to 8 and preferably 2 to 4 carbon units, with acetyl being exemplary.

U.S. Pat. No. 4,550,109 also describes biopterin compounds of the formula:

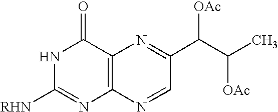

wherein R is a naturally occurring fatty acid, which can be saturated or unsaturated, and Ac═COCH₃. These biopterin compounds can be hydrogenated to form the corresponding tetrahydrobiopterins, which are useful according to the therapeutic methods of the invention. Exemplary chain lengths of the group R fatty acid range from C₁₀ to C₁₈ units. Exemplary compounds include 2N-Acetyl-1′,2′-di-O-Acetyl-L-Biopterin, 2-N-Decanoyl-1′,2′-di-O-acetyl-L-biopterin, 2-N-Palmitoyl-1′,2′-di-O-acetyl-L-biopterin, 2-N-Stearoyl-1′,2′-di-O-acetyl-L-biopterin, 2-N-Linoleyl-1′,2′-di-O-acetyl-L-biopterin, and 2-N-Phenylacetyl-1′,2′-di-O-acetyl-L-biopterin, and the corresponding tetrahydrobiopterins.

Preferably the 1′,2′-diacyl-(6R,S)-5,6,7,8-tetrahydro-L-biopterin or lipoidal tetrahydrobiopterin is selected as exhibiting at least 50% better oral bioavailability than BH4 ((6R)-5,6,7,8-tetrahydro-L-biopterin), for example, at least 15%, 20%, 25%, 30%, 35% or more oral bioavailability when taken on an empty stomach.

U.S. Pat. Nos. 4,752,573; 4,758,571; 4,774,244; 4,920,122; 5,753,656; 5,922,713; 5,874,433; 5,945,452; 6,274,581; 6,410,535; 6,441,038; 6,544,994; and U.S. Patent Publications US 20020187958; US 20020106645; US 2002/0076782; US 20030032616 (each incorporated herein by reference) each describe methods of administering BH4 compositions for various treatments. Each of those patents is incorporated herein by reference as providing a general teaching of methods of administering BH4 compositions known to those of skill in the art, that may be adapted by a person of ordinary skill in the art for the treatment described herein.

### 3. Pharmaceutical Formulations

The formulations described herein are preferably administered as oral formulations. Oral formulations are preferably solid formulations such as capsules, tablets, pills and troches, or liquid formulations such as aqueous suspensions, elixirs and syrups. The various forms of BH4 derivative prodrugs described herein can be directly used as powder (e.g., micronized particles), granules, suspensions or solutions, or they may be combined together with other pharmaceutically acceptable ingredients in admixing the components and optionally finely dividing them, and then filling capsules, composed for example from hard or soft gelatin, compressing tablets, pills or troches, or suspending or dissolving them in carriers for suspensions, elixirs and syrups. Coatings may be applied after compression to form pills.

Pharmaceutically acceptable ingredients are well known for the various types of formulation and can be, for example, binders such as natural or synthetic polymers, excipients, lubricants, surfactants, sweetening and flavoring agents, coating materials, preservatives, dyes, thickeners, adjuvants, antimicrobial agents, antioxidants and carriers for the various formulation types. Nonlimiting examples of binders useful in a composition described herein include gum tragacanth, acacia, starch, gelatin, and biological degradable polymers such as homo- or co-polyesters of dicarboxylic acids, alkylene glycols, polyalkylene glycols and/or aliphatic hydroxyl carboxylic acids; homo- or co-polyamides of dicarboxylic acids, alkylene diamines, and/or aliphatic amino carboxylic acids; corresponding polyester-polyamide-co-polymers, polyanhydrides, polyorthoesters, polyphosphazene and polycarbonates. The biological degradable polymers may be linear, branched or crosslinked. Specific examples include poly-glycolic acid, poly-lactic acid, and poly-d,l-lactide/glycolide. Other examples for polymers are water-soluble polymers such as polyoxaalkylenes (polyoxaethylene, polyoxapropylene and mixed polymers thereof, poly-acrylamides and hydroxylalkylated polyacrylamides, poly-maleic acid and esters or -amides thereof, poly-acrylic acid and esters or -amides thereof, poly-vinylalcohol und esters or -ethers thereof, poly-vinylimidazole, poly-vinylpyrrolidone, und natural polymers such as chitosan.

Nonlimiting examples of excipients (e.g. tableting excipients) useful in a composition described herein include phosphates such as dicalcium phosphate. Nonlimiting examples of lubricants use in a composition described herein include natural or synthetic oils, fats, waxes, or fatty acid salts such as magnesium stearate.

Surfactants for use in a composition described herein can be anionic, anionic, amphoteric or neutral. Nonlimiting examples of surfactants useful in a composition described herein include lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and polyoxyethylene stearate.

Nonlimiting examples of sweetening agents useful in a composition described herein include sucrose, fructose, lactose and aspartame. Nonlimiting examples of flavoring agents for use in a composition described herein include peppermint, oil of wintergreen or fruit flavors such as cherry or orange flavor. Nonlimiting examples of coating materials for use in a composition described herein include gelatin, wax, shellac, sugar or other biological degradable polymers. Nonlimiting examples of preservatives for use in a composition described herein include methyl or propylparabens, sorbic acid, chlorobutanol, phenol and thimerosal.

The BH4 prodrug described herein may also be formulated as effervescent tablet or powder, which disintegrates in an aqueous environment to provide a drinking solution. A syrup or elixir may contain the BH4 prodrug described herein, sucrose or fructose as sweetening agent a preservative like methylparaben, a dye and a flavoring agent.

Slow release formulations may also be prepared from the BH4 prodrug described herein in order to achieve a controlled release of the active agent in contact with the body fluids in the gastro intestinal tract, and to provide a substantially constant and effective level of the active agent in the blood plasma. Controlled release via dissolution control, diffusion control, and ion exchange are contemplated. The BH4 prodrug may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

While individual needs vary, determination of optimal ranges of effective amounts of each component is within the skill of the art. Such a dose may be administered in a single dose or it may be divided into multiple doses. While continuous, daily administration is contemplated, it may be desirable to cease the therapy when specific clinical indicators are improved to above a predetermined threshold level. Of course, the therapy may be reinitiated in the event that clinical improvement indicators deteriorate.

It is understood that the suitable dose of a composition according to the invention will depend upon the age, health and weight of the recipient, kind of concurrent treatment, if any, frequency of treatment, and the nature of the effect desired (i.e., the amount of decrease in pulmonary pressures desired). The frequency of dosing also is dependent on pharmacodynamic effects on arterial oxygen pressures. However, the most preferred dosage can be tailored to the individual subject, as is understood and determinable by one of skill in the art, without undue experimentation. This typically involves adjustment of a standard dose, e.g., reduction of the dose if the patient has a low body weight.

As discussed above, the total dose required for each treatment may be administered in multiple doses or in a single dose. The BH4 derivative compositions may be administered alone or in conjunction with other therapeutics directed to the disease or directed to other symptoms thereof.

For solutions, one will generally desire to employ appropriate salts and buffers to render the BH4 derivative suitable for uptake. Aqueous compositions of the invention will comprise an effective amount of the BH4 derivative dissolved or dispersed in a pharmaceutically acceptable carrier or aqueous medium. Such compositions may be administered orally or via injection.

The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional medium or agent is incompatible with the therapeutic compositions, its use in therapeutic compositions is contemplated. Supplementary active ingredients also can be incorporated into the compositions. In exemplary embodiments, the formulation may comprise corn syrup solids, high-oleic safflower oil, coconut oil, soy oil, L-leucine, calcium phosphate tribasic, L-tyrosine, L-proline, L-lysine acetate, DATEM (an emulsifier), L-glutamine, L-valine, potassium phosphate dibasic, L-isoleucine, L-arginine, L-alanine, glycine, L-asparagine monohydrate, L-serine, potassium citrate, L-threonine, sodium citrate, magnesium chloride, L-histidine, L-methionine, ascorbic acid, calcium carbonate, L-glutamic acid, L-cystine dihydrochloride, L-tryptophan, L-aspartic acid, choline chloride, taurine, m-inositol, ferrous sulfate, ascorbyl palmitate, zinc sulfate, L-carnitine, alpha-tocopheryl acetate, sodium chloride, niacinamide, mixed tocopherols, calcium pantothenate, cupric sulfate, thiamine chloride hydrochloride, vitamin A palmitate, manganese sulfate, riboflavin, pyridoxine hydrochloride, folic acid, beta-carotene, potassium iodide, phylloquinone, biotin, sodium selenate, chromium chloride, sodium molybdate, vitamin D₃ and cyanocobalamin. The amino acids, minerals and vitamins in the supplement should be provided in amounts that provide the recommended daily doses of each of the components.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional medium or agent is incompatible with the active ingredient, its use in the therapeutic compositions is contemplated. Supplementary active ingredients also can be incorporated into the compositions.

The active compositions of the present invention include classic pharmaceutical preparations of BH4 derivative, which have been discussed herein as well as those known to those of skill in the art. Moreover, the BH4 derivative can be incorporated into dietary supplements, such as the low-phenylalanine or phe-free amino acid compositions, infant formula, and special PKU foods. Administration of these compositions according to the present invention will be via any common route for dietary supplementation. The protein is preferably administered orally, as is the BH4 derivative.

In certain embodiments, it is contemplated that the BH4 derivatives used for the treatment of BH4-related diseases are formulated as an inhalable formulation for administration through inhalation. As such, the BH4 derivatives may be prepared as an aerosol formulation. Methods to the treatment using inhalable compositions are known to those of skill in the art and are described, for example, in U.S. Pat. No. 6,756,033 (incorporated herein by reference), which provides a teaching of delivery of prostaglandin preparations by inhalation. The inhalation techniques described in the aforementioned patent for prostaglandins also will be useful in producing inhalable preparations of a BH4 derivatives described herein. In addition, it is contemplated that a combined administration of a BH4 derivative and a prostaglandin preparation can be used as effective treatments.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The BH4 derivative compositions may be prepared as pharmaceutical forms suitable for injectable use. Such compositions include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the BH4 derivative compounds described herein in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′ dibenzylethylenediamine, chloroprocaine, choline, diethanolamine, dicyclohexylamine, ethylenediamine, N methylglucamine, and procaine.

Pharmaceutically acceptable acid addition salts include inorganic or organic acid salts. Examples of suitable acid salts include the hydrochlorides, acetates, citrates, salicylates, nitrates, and phosphates. Other suitable pharmaceutically acceptable salts are well known to those skilled in the art and include, for example, acetic, citric, oxalic, tartaric, or mandelic acids, hydrochloric acid, hydrobromic acid, sulfuric acid or phosphoric acid; with organic carboxylic, sulfonic, sulfo or phospho acids or N substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4 aminosalicylic acid, 2 phenoxybenzoic acid, 2 acetoxybenzoic acid, embonic acid, nicotinic acid or isonicotinic acid; and with amino acids, such as the 20 alpha amino acids involved in the synthesis of proteins in nature, for example glutamic acid or aspartic acid, and also with phenylacetic acid, methanesulfonic acid, ethanesulfonic acid, 2 hydroxyethanesulfonic acid, ethane 1,2 disulfonic acid, benzenesulfonic acid, 4 methylbenzenesulfoc acid, naphthalene 2 sulfonic acid, naphthalene 1,5 disulfonic acid, 2 or 3 phosphoglycerate, glucose 6 phosphate, N cyclohexylsulfamic acid (with the formation of cyclamates), or with other acid organic compounds, such as ascorbic acid.

Specifically, BH4 derivative salts with inorganic or organic acids are preferred. Nonlimiting examples of alternative BH4 derivative salts forms includes salts of acetic acid, citric acid, oxalic acid, tartaric acid, fumaric acid, and mandelic acid.

The frequency of BH4 derivative dosing can depend on the pharmacokinetic parameters of the agent and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042) pp 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood levels of Phe in conjunction with relevant dose response data. The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As more studies are conducted, further information will emerge regarding appropriate dosage levels and duration of treatment for specific diseases and conditions.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey, ducks, and geese.

In certain aspects of the present invention, all the necessary components for the treatment of disease using BH4 derivative either alone or in combination with another agent or intervention traditionally used for the treatment of such disease may be packaged into a kit. Specifically, the present invention provides a kit for use in the therapeutic intervention of the disease comprising a packaged set of medicaments that comprise a BH4 derivative described herein as well as buffers and other components for preparing deliverable forms of said medicaments, and/or devices for delivering such medicaments, and/or any agents that are used in combination therapy with such BH4-based medicaments, and/or instructions for the treatment of the disease packaged with the medicaments. The instructions may be fixed in any tangible medium, such as printed paper, or a computer-readable magnetic or optical medium, or instructions to reference a remote computer data source such as a world wide web page accessible via the internet.

### III. Factors that Alter BH4 Synthesis

The present invention contemplates a method of treating a disease or disorder comprising administering to said subject a composition comprising a BH4 derivative alone or in combination with a therapeutic agent wherein said administration is effective in alleviating the dysfunction of said subject as compared to said dysfunction in the absence of said BH4 derivative.

One embodiment of the invention includes one or more agents that increase BH4 levels by increasing the expression or synthesis or the activity of the enzymes in the BH4 synthetic pathway including the first and the rate-controlling enzyme GTPCH1, PTPS and SR. In a preferred embodiment of the invention, BH4 synthesis is increased by increasing the expression of GTPCH1 expression by the use of any one or more cyclic adenosine monophosphate (cAMP) analogs or agonists including forskolin, 8-bromo cAMP or other agents that function to increase cAMP mediated cell signaling, for example, cytokines and growth factors including interleukin-1, interferon-gamma (IFN-gamma), tumor necrosis factor alpha (TNF-alpha), c-reactive protein, HMG-CoA-reductases (statins like atorvastatin) nerve growth factor (NGF), epidermal growth factor (EGF), hormones including adrenomedullin and estradiol benzoate, and other compounds such as NADPH and NADPH analogs, caffeine, cyclosporine A, methyl-xanthines including 3-isobutyl-1-methyl xanthine, theophylline, reserpine, or hydrogen peroxide.

It is well established in the art that the phosphodiesterases degrade the 3′5′-cyclic nucleotides such as cGMP and cAMP. cAMP is a known activator of GTPCH1 the rate controlling enzyme for BH4 synthesis the required co-factor for eNOS. Inhibitors of phosphodiesterases family, thus, have a secondary activating effect on BH4-synthetic enzyme GTPCH1. One embodiment of invention therefore relates to increasing GTPCH1 levels by inhibiting the degradation of 3′5′-cyclic nucleotides using inhibitors of the eleven phosphodiesterases families (PDE1-11) including PDE1, PDE3, PDE5. The PDE inhibitors of the present invention include Viagra/sildanfil, cialis/tadalfil, vardenafil/levitra, udenafil, 8-Methoxymethyl-IBMX, UK-90234, dexamethasone, hesperetin, hesperedins, Irsogladine, vinpocetine, cilostamide, rolipram, ethyl beta-carboline-3-carboxylate (beta-CCE), tetrahydro-beta-carboline derivatives, 3-O-methylquercetin and the like.

Another embodiment of the invention relates to increasing the levels of BH4 by increasing the levels of BH4-synthesizing enzymes by gene therapy or endothelium-targeted delivery of polynucleotides of the synthetic machinery of BH4. Patents filed claiming BH4-synthesizing genes to be used in gene therapy include U.S. Patent Application Publication No. 20030198620. Yet another embodiment of the invention relates to increasing the levels of BH4 by supplementation with BH4-synthesizing enzymes GTPCH1, PTPS, SR, PCD, DHPR and DHFR. It is contemplated that by BH4-synthesizing enzymes, it is asserted that all natural and unnatural forms of the enzymes including mutant of the proteins active and inactive are included.

Another embodiment of the invention relates to increasing BH4 levels by diverting the substrate 7,8-dihydroneopterin triphosphate towards BH4 synthesizing enzyme PTPS instead of alkaline phosphatase (AP) by inhibiting AP activity. The agents or compounds that inhibit the activity of AP include phosphate analogs, levamisole, and L-Phe. Another embodiment of the invention relates to agents or compounds that inhibit alkaline phosphatase includes the small inhibitory RNA (siRNA), antisense RNA, dsDNA, small molecules, neutralizing antibodies, single chain, chimeric, humanized and antibody fragments to inhibit the synthesis of alkaline phosphatase.

Another embodiment of the invention includes agents or compounds that enhance the activity of catalysts or cofactors needed for the synthesis of enzymes of the de novo synthesis pathway of BH4 synthesis.

Another embodiment of the invention includes agents or compounds that prevent the degradation of the enzymes needed for the synthesis of BH4. Yet another embodiment of the invention includes agents or compounds that prevent the degradation of the catalysts needed for the synthesis of BH4 and its synthetic enzymes including GTPCH1, PTPS and SR.

Another embodiment of the invention relates to increasing BH4 levels by inhibiting the feedback modulation of the GTPCH1/GFRP complex by BH4. A preferred embodiment of the invention relates to agents or compounds that inhibit the binding of BH4 to the GTPCH1/GFRP complex, thereby preventing the feedback inhibition by BH4. Agents or compounds of this invention include competitive inhibitors such as alternate forms of BH4 with altered affinities for the complex, structural analogs etc. Still another embodiment of the invention includes agents or compounds that enhance the binding of L-phenylalanine to CTPCH1/GFRP inducing the synthesis of BH4. Another embodiment of the invention includes agents or compounds that increase the levels of L-Phe such as precursors of L-Phe.

Yet another embodiment of the invention relates to agents or compounds that modulate the activity or the synthesis of GFRP. A preferred embodiment of the invention includes agents or compounds that inhibit the activity of GFRP. Another embodiment of the invention includes the use of siRNA, small molecules, antibodies, antibody fragments and the like to inhibit the synthesis of GFRP.

Another embodiment of the invention relates to increasing the levels of BH4 by increasing the reduction of BH2 via the salvage pathway. In vivo, BH4 becomes oxidized to BH2. BH2 which exist as the quinoid form (qBH2) and as the 7,8-dihydropterin which is reduced to BH4 by DHPR and DHFR respectively. A preferred embodiment of the invention relates to increasing the regeneration or salvage of BH4 from BH2 by modulating the activity and synthesis of the enzymes PCD, DHPR and DHFR using agents or compounds that pathway NADPH, thiols, perchloromercuribenzoate, hydrogen peroxide and the like.

Another embodiment of the invention relates to increasing the levels of active BH4 by decreasing the oxidation of BH4 using agents or compounds such as antioxidants including ascorbic acid (vitamin C), vitamin E, tocopherols (e.g vitamin A), selenium, beta-carotenes, carotenoids, flavones, flavonoids, folates, flavones, flavanones, isoflavones, catechins, anthocyanidins, chalcones etc. US patents and patent application publications U.S. Pat. No. 6,544,994, US20050119270, US20030007961, and US20020052374 describe the use of BH4 and antioxidant.

Yet another embodiment of the invention includes agents or compounds that are the precursors of BH4 including guanosine triphosphate, 7,8-dihydro-neopterin triphosphate and 6-pyrovoyl tetrahydropbiopterin.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

BH4 derivatives are prepared as described in U.S. Pat. Nos. 4,540,783 and/or 4,550,109.

### Example 2

This example describes an assay for metabolic stability and allows comparison of the stability of a BH4 derivative versus that of BH4.

Test compounds (10 uM) are incubated with mouse, rat and human liver microsomes (protein concentration of 0.5 mg/mL) and 1 mM NADPH in phosphate buffer at 37° C. Experiments are conducted in triplicate. The incubations are initiated by the addition of the microsomes and quenched by the addition of an equal volume of methanol. Samples are taken at two to three timepoints (typically at time zero, 30 minutes, 60 minutes) for analysis. The appropriate positive and negative control incubations are performed. The quantitation of the disappearance of the test compound or % turnover of the test article is determined utilizing LC-MS/MS.

### Example 3

This example describes an assay for solubility and allows comparison of the solubility of a BH4 derivative versus that of BH4. The test articles are dissolved in DMSO and serially diluted in phosphate buffered saline pH 7.4 (PBS) in a 96 well plate. The diluted compounds have a final concentration range of 1 to 1000 mg/mL and contain ≦1% DMSO. After a 30-minute incubation at room temperature, precipitation is measured by detecting light scattering on a Lab Systems nephelometer. Solubility is determined by comparing the NU (nephelometer units) of four replicates of a sample concentration to the NU of the solvent blank wells. Insolubility is defined as the concentration at which the blank corrected NU is significantly greater than the solvent blank. A 1% difference calculated by Student's T Test is considered to be significant.

### Example 4

This example describes the permeability screen using Caco-2 cell monolayers and allows comparison of the permeability of a BH4 derivative versus that of BH4. Monolayer cultures of Caco-2 cells, suitable for investigation of compound permeability, are grown on either 24- or 96-well polycarbonate membrane inserts for 21 to 30 days. The monolayers are maintained at 37° C. in a 5% CO₂ atmosphere at 95% relative humidity until confluent. The maturity and membrane integrity of the monolayers are confirmed by measurement of the trans-epithelial electrical resistance (TEER) or the apparent permeability of the fluorescent marker compound lucifer yellow.

Apparent permeabilities of a series of test and selected marker compounds are determined in duplicate at a single concentration of 10 mM in the apical to basolateral direction. The transport investigations are initiated by the addition of the test compound to the apical compartment and the plates are maintained under culture conditions during the course of the experiment. The basolateral compartments following 30 and 60 minutes of exposure and the final apical compartments are collected and analyzed for test compound content by LC-MS/MS. The recovery and apparent permeability of each test compounds are calculated from these data. Appropriate controls are included to characterize the monolayers. The transport experiment from the basolateral to the apical side will also be performed in the presence and absence of a P-gp inhibitor such as verapamil.

### Example 5

This example describes a study of the bioavailability/pharmacokinetics profile as performed with a BH4 derivative and BH4. The purpose of pharmacokinetic studies is to provide information on systemic exposure of a drug and any metabolites. This data can be used to explain pharmacological or toxicological issues and can also aid in the design of toxicokinetic studies. Pharmacokinetic parameters, such as AUC, half-life, clearance and volume of distribution, are also determined.

The purpose of the study is to evaluate the potential oral availability of the test compounds, estimate the pharmacokineric parameters via statistical approximation, and compare such values to that obtained with unaltered BH4. A simple, non-GLP extraction and LC-MS/MS analytical method is developed for plasma analysis. The formula and structural information of the test compounds is reviewed and plasma stability is presumed. If the test compound is unstable in the plasma, methods are modified as necessary. The study involves three healthy rats of either sex per test compound. Dose formulations are prepared by solution or suspension of the test compounds in water, saline, Tween, PEG, or similar vehicle. For each test compound, three rats are dosed at one time via oral gavage and blood collected at four timepoints (1, 2, 4, 8 hours). Concentrations of drug in plasma are measured using LC-UV or LC-MS(/MS) to define plasma concentration-time curve. Pharmacokinetic parameters such as Cmax, Tmax, and Area Under the Curve (AUC) are estimated using WinNonlin (Pharsight Corp.).

If mice are used instead of rats, 12 mice will be used for each test compound. Samples are taken from three mice per timepoint, and pharmacokinetics will be estimated using mean plasma concentration data per timepoint.

Using in vitro metabolism data, concentrations of major metabolites can also be estimated. Collecting excreta during the study period and analysis of these samples for parent and metabolites gives an estimate of elimination.

### Example 6

This example allows for comparison of the pharmacokinetics of the BH4 derivative versus that of BH4 following single oral administration in rats.

Single doses of BH4 (10 and 100 mg/kg) were administered orally to a first group of male Sprague Dawley rats (6 weeks old) under fasting conditions. Single doses of Compound I were administered orally to a first group of male Sprague Dawley rats (6 weeks old) under fasting conditions.

With respect to the administered BH4, the maximum total biopterin concentrations in plasma 2 hrs and 1 hr post-dosing were 108 ng/ml (i.e., about 3 fold the endogenous level) and 1227 ng/ml (i.e., about 30 fold the endogenous level), respectively. Thereafter, biopterin had an elimination half-life (t_{1/2}) of about 1.1 hr, returning to the endogenous level 9 hrs post-dosing for the 10 mg/kg dose and 24 hrs post-dosing for the 100 mg/kg dose. The bioavailability (F) after a 10 and 100 mg/kg oral administration were 6.8% and 11.8%, respectively, based on the area under the plasma concentration-time curve (AUC) obtained by subtracting the endogenous level during a 10 mg/kg intravenous administration. The ratio of reduced biopterin to total biopterins in plasma (i.e., the reduced-form ratio) was relatively static (73%-96%).

The BH4 derivative is similarly tested and evaluated. The AUC and the peak (Cmax) is expected to be better than that of BH4, due to its increased bioavailability. The bioavailability is at least 15, 20, or 30% or above, and up to 500% above that of BH4, depending upon the derivative.

### Example 7

The example allows comparison of the pharmacokinetics of sapropterin and the BH4 derivative after a single oral administration in groups of cynomolgus monkeys.

A single dose of sapropterin (10 mg/kg) was administered orally to a first group of female cynomolgus monkeys (3/group) under fasting conditions. A single dose of Compound I (10 mg/kg) was administered orally to second group of female cynomolgus monkeys (3/group) under fasting conditions.

With respect to the administered sapropterin, the total plasma biopterin concentration (ΔC) reached its maximum value 3 hrs post-dosing (344 ng/ml, approximately 20× endogenous levels). The plasma elimination half-life of biopterin was approximately 1.4 hrs, returning to the endogenous level within 24 hrs post dosing. The ratio of reduced biopterin to total biopterins was nearly constant during the test period. The bioavailability (F) following a 10 mg/kg oral administration to female monkeys was about 9%.

BH4 derivative is similarly tested and evaluated in female cynomolgus monkeys.

### Example 8

This example provides a protocol for clinical evaluation of a BH4 derivative versus BH4, by specifically studying the absorption in the gastrointestinal tract.

Gastrointestinal absorption is evaluated in humans in a blinded cross-over study

Unless otherwise stated, subjects are given either tetrahydrobiopterin (BH4) or a BH4 derivative prepared as described in Example 1, at a dose of 1, 5, and 10 mg/kg after a fast of 10 hours. Blood samples are collected in heparinized vials at 0, 0.5, 1, 2, 3, 4, 6, 8, k 12, 24, 48, 72, 96, 120, 144 h post dose. For a single dose and relative bioavailability study, plasma samples are also collected 0.25, 0.75 and 1.5 hours after administration and assayed for total biopterin to evaluate the site of gastrointestinal absorption of either BH4 or the BH4 derivative.

Subjects are given a 1, 5, and 10 mg/kg oral or intravenous dose of either BH4 or the BH4 derivative, followed by serial measurements of plasma total biopterin concentration to determine the rate of BH4 or the BH4 derivative absorption from the gastrointestinal tract from the area under the plasma total biopterin concentration increase (ΔCp)-time curve (ΔAUC). It is anticipated that a lower dose of BH4 will be required when administered intravenously in comparison with BH4 administered orally. to achieve the same level of bioavailability. For example, it may require 10 mg/kg of BH4 given orally to achieve the same level of bioavailability as 1 mg/kg BH4 administered intravenously. Because the BH4 derivative exhibits enhanced bioavailability, it may require only 2.5 mg/kg of the BH4 derivative to achieve the same level of bioavailability as a 1 mg/kg IV dose of BH4. to achieve the same percent bioavailability.

The rate of BH4 or BH4 derivative absorption from the gastrointestinal tract is estimated from the area under the plasma total biopterin concentration increase (ΔCp)-time curve (ΔAUC) after the administration BH4 or BH4 derivative using the following formulas:

Absorption rate(%)=(ΔAUC after p.o. dose/ΔAUC after i.v. dose)×(i.v. dose/p.o. dose×100)

Some derivatives of BH4 may require a longer duration to release the active BH4. Thus, a measurement of free or released BH4 alone in the blood may not accurately reflect the total amount of BH4 that could be available for treatment. Hence, a measurement of the total concentration of the derivative and BH4 together is required to accurately or more precisely determine the level of BH4 in the blood for the purposes of evaluating bioavailability and comparing bioavailability of the derivative and BH4.

### Example 9

This example provides a protocol for administration of BH4 derivative to humans with elevated serum Phe levels.

A BH4 derivative prepared as in Example 1 is administered to humans with elevated blood levels of phenylalanine (>600 μmol/L). Criteria for inclusion in an exemplary clinical study include (1) baseline blood Phe levels of >600 μmol/L, (2) age of at least 8 years. Criteria for exclusion from the study include (1) pregnancy or breastfeeding, (2) concurrent diseases or conditions that require medication or treatment, (3) concurrent treatment with any drug known to inhibit folate synthesis, and (4) treatment with any investigational drug within 30 days. Each of the patients is optionally also identified as having a mutation in the phenylalanine hydroxylase (PAH) gene. Study subjects undergo baseline assessments, including medical history with assessment of phenylketonuria (PKU) or hyperphenylalaninemia (HPA) related signs and symptoms, physical examinations, vital signs, serum amino acid (i.e., phenylalanine, tyrosine, and tryptophan) blood levels, and routine laboratory tests (chemistry, hematology, and urinalysis) before inclusion in the study.

Drugs known to inhibit folate synthesis such as bactrim, methotrexate, or 5-FU are not permitted to be administered during the study. Before initiation of dosing, a 7 day washout period is required for any drugs known to inhibit folate synthesis. No investigational drugs are permitted to be taken during study participation or within 30 days prior to study enrollment.

Within a maximum of 4 weeks following the completion of baseline assessments, eligible subjects begin the first stage of the study. Single ascending doses of 1, 2, 4, 5 and/or 8 mg/kg of the BH4 derivative are administered orally, with a washout period of at least 7 days between each dose, and subjects are monitored 24 hours after each dose. Subjects undergo a safety assessment and blood amino acid (i.e, phenylalanine, tyrosine, and tryptophan) level measurements before and 24 hours after each BH4 derivative dose. Blood pressure is measured 30 minutes and 1 hour after each dose. Safety assessments include physical examinations, vital signs, serial assessment of PKU or HPA related signs and symptoms, recording of adverse events, and monitoring of changes in laboratory parameters (chemistry, hematology, and urinalysis). Subjects are instructed to continue their usual diet without any modification, and to record daily intake of food and beverages throughout the study.

Alternatively, or in addition to the single dose studies, subjects receive the prescribed dosage of BH4 derivative daily in an oral dosage form, for a total of 7 days. After a washout period of at least 7 days, each subject may receive a higher dose of BH4 derivative for another 7 days. Subjects are monitored before dosing, at 24 and 72 hours after first dose, and on the 7th day of dosing at each of the dose levels. Monitoring includes a safety assessment as described above, measurement of serum blood amino acid (i.e, phenylalanine, tyrosine, and tryptophan) levels and evaluation of phenylalanine and tyrosine oral intake. Subjects are instructed to continue their usual diet without any modification, and to record daily intake of food and beverages throughout the study.

Administration of the BH4 derivative is expected to provide a decline in blood Phe levels compared to baseline, in a dose-dependent manner.

### Example 10

This example provides a protocol for administration of BH4 derivative to patients suffering from neuropsychiatric disorders, generally according to the protocol disclosed in U.S. Pat. No. 6,200,758.

Subjects receive the prescribed dosage of BH4 derivative daily in an oral dosage form, for a total of 2, 4, 6 or 8 weeks. Patients are given a complete neurological and psychiatric assessment to identify clinical symptoms of disease.

If appropriate, psychiatric symptoms are monitored by use of the Brief Psychiatric Rating Scale (Overall, J. E., “The Brief Psychiatric Rating Scale” Psychol. Rep., 1962; 10: 799-812). Decreases in, for example, the Anxiety-Depression factor and/or Thought Disturbance factor may be observed. The symptoms included in the Anxiety-Depression factor are the symptoms of somatic concern, anxiety, guilt feelings and depressive mood, and those of the Thought Disturbance factor are the symptoms of conceptual disorganization, grandiosity, hallucinatory behavior, and unusual thought content.

Parkinson's symptoms may be assessed, including range of motion of extremities, neck, shoulder or ankle, strength, rigidity, sensation, coordination, flexibility, tremor, reciprocal motion, hand writing, hand prehensions, isolated finger control, grip strength, activities of daily living tasks, cognitive skills, moving, walking, gait, and balance (optionally using Tinetti's Balance Test).

Other assessment techniques may be used, including the Global Deterioration Scale (GDS), Brief Cognitive Rating Scale (BCRS), or Functional Assessment Staging (FAST).

All publications cited above are, in relevant part, incorporated herein by reference. The citation of any publication is not to be construed as an admission that it constitutes prior art relative to the disclosed invention.

The foregoing description is given for clearness of understanding only, and no unnecessary limitations should be understood therefrom, as modifications within the scope of the invention may be apparent to those having ordinary skill in the art.

1. A method of treating a tetrahydrobiopterin (BH4)-responsive metabolic disorder or neuropsychiatric disorder comprising administering a BH4 derivative prodrug orally to a subject in need thereof at a dose of less than 5 mg/kg/day.

2. The method of claim 1 wherein the BH4 derivative prodrug is a diacyl tetrahydrobiopterin, a diacetyl tetrahydrobiopterin, or a lipoidal tetrahydrobiopterin.

3. The method of claim 1, wherein the dose is 0.1 mg/kg/day to 4 mg/kg/day.

4-5. (canceled)

6. The method of claim 1, wherein the subject has a metabolic disorder selected from the group consisting of hyperphenylalanemia, mild phenylketonuria, moderate phenylketonuria, severe phenylketonuria, atypical or malignant phenylketonuria associated with BH4 deficiency, hyperphenylalanemia associated with liver disorder, and hyperphenylalanemia associated with malaria.

7-8. (canceled)

9. The method of claim 1, wherein the subject has a neuropsychiatric disorder selected from the group consisting of Parkinson's disease, Alzheimer's disease, schizophrenia, schizophreniform disorder, schizoaffective disorder, brief psychotic disorder, delusional disorder, shared psychotic disorder, psychotic disorder due to a general medical condition, substance-induced psychotic disorder, other psychotic disorders, tardive dyskinesia, Machado-Joseph disease, spinocerebellar degeneration, cerebellar ataxia, dystonia, chronic fatigue syndrome, acute or chronic depression, chronic stress syndrome, fibromyalgia, migraine, attention deficit hyperactivity disorder, bipolar disease, and autism.

10. The method of claim 1, wherein the BH4 derivative prodrug is a di-ester of BH4 modified at the BH4 hydroxyl sites.

11. The method of claim 10, wherein the ester is selected from the group consisting of formyl, acetyl, propyl, and butyral.

12. (canceled)

13. The method of claim 1, further comprising administering a second therapeutic agent, said therapeutic agent capable of (1) enhancing activity, expression, or de novo biosynthesis of BH4, (2) reducing degradation of BH4 or derivatives thereof, (3) stabilizing BH4 or derivatives thereof, or (4) combinations thereof.

14. The method of claim 13, wherein the therapeutic agent (1) increases activity or expression of guanosine triphosphate cyclohydrolase I (GTPCH1), 6-pyruvoyltetrahydropterin synthase (PTPS) or sepiapterin reductase; (2) increases GTPCH1 levels by inhibiting degradation of 3′5′-cyclic nucleotides and is an inhibitor of a phosphodiesterase; (3) increases BH4 levels by diverting the substrate 7,8-dihydroneopterin triphosphate towards BH4 synthesizing enzyme PTPS instead of alkaline phosphatase (AP) by inhibiting AP activity; (4) stabilizes BH4 or a derivative thereof by decreasing oxidation of BH4 or a derivative thereof; (5) modulates GTPCH feedback regulatory protein (GFRP); (6) an alternate form of BH4 having an altered affinity for a GTPCH1/GFRP complex; (7) inhibits GFRP synthesis and is selected from the group consisting of siRNA, a small molecule, an antibody, and an antibody fragment; (8) enhances binding of L-phenylalanine to GTPCH1/GFRP inducing the synthesis of BH4; or (9) is a precursor of BH4 and is selected from the group consisting of guanosine triphosphate, 7,8-dihydro-neopterin triphosphate and 6-pyrovoyl tetrahydropbiopterin.

15. The method of claim 13, wherein the therapeutic agent increases expression of GTPCH1 and is selected from the group consisting of a cyclic adenosine monophosphate (cAMP) analog or agonist, forskolin, 8-bromo cAMP, an agent that functions to increase cAMP mediated cell signaling, a cytokine, a growth factor, interleukin-1, interferon-gamma (IFN-γ), tumor necrosis factor alpha (TNF-α), c-reactive protein, HMG-Co A-reductase (statins like atorvastatin), nerve growth factor (NGF), epidermal growth factor (EGF), a hormone, adrenomedullin, estradiol benzoate, NADPH, a NADPH analog, caffeine, a cyclosporine A methyl-xanthine, 3-isobutyl-1-methyl xanthine, theophylline, reserpine, hydrogen peroxide, and mixtures thereof.

16. (canceled)

17. The method of claim 13, wherein the therapeutic agent is selected from the group consisting of sildanafil, tadalafil, vardenafil, udenafil, 8-methoxymethyl-IBMX, UK-90234, dexamethasone, hesperetin, hesperedin, Irsogladine, vinpocetine, cilostamide, rolipram, ethyl beta-carboline-3-carboxylate (beta-CCE), a tetrahydro-beta-carboline derivative, 3-O-methylquercetin, and mixtures thereof.

18. The method of claim 13, wherein the therapeutic agent is a BH4-synthesizing enzyme and is selected from the group consisting of guanosine triphosphate cyclohydrolase I (GTPCH1), 6-pyruvoyltetrahydropterin synthase (PTPS), sepiapterin reductase (SR), PCD, DHPR and DHFR.

19. (canceled)

20. The method of claim 13, wherein the therapeutic agent is selected from the group consisting of a phosphate analog, levamisole, L-phenylalanine, small inhibitory RNA (siRNA), antisense RNA, double stranded DNA (dsDNA), neutralizing antibodies, and single chain, chimeric, humanized antibody fragments which inhibit the synthesis of alkaline phosphatase.

21. (canceled)

22. The method of claim 13, wherein the therapeutic agent is selected from the group consisting of ascorbic acid (vitamin C), alpha tocopherol (vitamin E), tocopherols (e.g vitamin A), selenium, a beta-carotene, a carotenoid, a flavone, a flavonoid, a folate, a flavanone, an isoflavone, a catechin, an anthocyanidin, and a chalcone.

23. The method of claim 22, wherein the therapeutic agent is a folate and is selected from the group consisting of a folate precursor, a folic acid, a folate derivative, tetrahydrofolate, 5-formyl-(6S)-tetrahydrofolic acid or a salt thereof, 5-methyl-(6S)-tetrahydrofolic acid or a salt thereof, 5,10-methylene-(6R)-tetrahydrofolic acid or a salts thereof, 5,10-methenyl-(6R)-tetrahydrofolic acid or a salt thereof, 10-formyl-(6R)-tetrahydrofolic acid, 5-formimino-(6S)-tetrahydrofolic acid or a salt thereof, (6S)-tetrahydrofolic acid or a salt thereof, and combinations thereof.

24-27. (canceled)

28. The method of claim 13, wherein the therapeutic agent is a precursor of BH4 and is selected from the group consisting of guanosine trisphosphate, 7,8-dihydro-biopterin triphosphate and 6-pyrovoyl tetrahydrobiopterin.

29-31. (canceled)
